(19) United States
(12) Patent Application Publication
(10) Pub. No.: US 2015/0353515 A1
(43) Pub. Date: Dec. 10, 2015

(54) Novel analogs of resveratrol

(71) Applicants: Wen-Hsin Huang, Taipei (TW); Chien-Ming Huang, Taipei (TW); An-Rong Lee, Taipei (TW); Jiajiu Shaw, Ann Arbor, MI (US)

(72) Inventors: Wen-Hsin Huang, Taipei (TW); Chien-Ming Huang, Taipei (TW); An-Rong Lee, Taipei (TW); Jiajiu Shaw, Ann Arbor, MI (US)

(21) Appl. No.: 14/297,806

(22) Filed: Jun. 6, 2014

(51) Int. Cl. C07D 277/64

(52) U.S. Cl. CPC C07D 277/64

(57) ABSTRACT

For the reported resveratrol analogs, the (E)-stilbene core structure is either maintained intact or only slightly modified. We use a different approach and introduced a novel heterocyclic benzothiazolium moiety into the (E)-stilbene core to form a series of novel resveratrol analogs for treating angiogenic- and/or inflammation-related disorders.

### FIELD OF THE INVENTION

This invention is related to the design and synthesis of novel resveratrol analogs and methods of treating angiogenic- and/or inflammation-related disorders.

### INTRODUCTION

Resveratrol (FIG. 1), 3,5,4′-trihydroxy-trans-stilbene, is a polyphenol found in grapes, blue berries, peanuts, and a number of other plants. Resveratrol exhibits a variety of useful biological properties including antileukemic, antibacterial, antifungal, antiplatelet aggregation, coronary vasodilator, antioxidative, and antiinflammatory activities and so on. In the field of arthritis, it was recently reported that resveratrol inhibits the proliferation of synoviocytes in rheumatoid arthritis (RA) in vitro. It was suggested that intra-articular injection of resveratrol may protect cartilage against the development of experimentally induced inflammatory arthritis. Resveratrol has been reported to inhibit nitric oxide production by lipopolysaccharide-activated brain microglia. Resveratrol revealed effectively anti-angiogenic action as well.

Because of the biologically beneficial effects of resveratrol, many attempts have been made to modify its structure in order to further improve its biological activities and water-solubility. A number of hetercyclic resveratrol analogs have been reported and some of them have shown interesting biological activities (References 1-5). However, for most of the reported analogs, the (E)-stilbene core structure is either maintained intact or only slightly modified.

### DETAILED DESCRIPTION OF THE INVENTION

We hypothesized that it may not be necessary to maintain the core structure of resveratrol in order to improve its biological activities. Based on this hypothesis, we introduced a novel heterocyclic benzothiazolium moiety into the (E)-stilbene core and investigated their biological properties as compared with resveratrol.

### Synthesis

Research chemicals were purchased from Sigma-Aldrich or Alfa Aesar (at least 95% purity) and used without further purification. The reference compound, resveratrol (99% GC purity), was purchased from Sigma-Aldrich. Reactions were monitored by thin-layer chromatography (TLC) on silica gel plates (60 F254; Merck) visualizing with ultraviolet light or iodine. Melting points were taken in open capillary tubes on a Buchi-530 melting point apparatus and are uncorrected. ¹H-NMR spectra were determined on a Varian Gemini-300 NMR instrument. Chemical shifts (8) are reported in parts per million (ppm) relative to tetramethylsilane (TMS) as an internal standard; coupling constants (J) are shown in hertz (Hz) and signals are described as s (singlet), d (doublet), t (triplet), and m (multiplet); IR spectra were recorded on a Perkin-Elmer FTIR 1610 series infrared spectrophotometer in KBr discs. Fast atom bombardment (FAB) mass spectra were recorded using a JEOL-SX102A (GC/LC/MS) spectrometer. Only peaks of significant relative intensity are presented in the form of m/z (intensity relative to base peak). Elemental analyses for carbon, hydrogen, nitrogen and sulfur were performed in the Instrument Center of the National Science Counsel at the National Taiwan University using HERAEUS VarioEL analyzer.

### Example 1

### Synthesis of Compound 2

### 3-Ethyl-2-methylbenzothiazolium bromide

Two methods were utilized for the synthesis of 3-ethyl-2-methylbenzothiazolium bromide (2): (a) in a CEM Discover microwave reactor, and (b) by a traditional reflux, resulting in 57% and 32% yields, respectively.

The starting material, 2-Methylbenzothiazole (1.5 g, 10 mmol), was reacted with ethyl bromide (1.5 ml, 20 mmol) over 5 h in a special closed glass tube (explosive-proof) using CEM microwave reactor under 250 watts, 120° C., to obtain the product as precipitate. Filter and wash the precipitate with ethyl ether or dichloromethane to obtain the pure product (1.47 g, 57% yield). The product is soluble in acetone and in water. Alternative method without the microwave reactor: 2-methylbenzothiazole (1.5 g, 10 mmol) was reacted with ethyl bromide (15 ml, 200 mmol) in 100 ml of acetonitrile (as the solvent) under reflux over 72 h to obtain the product as precipitate. Filter and wash the precipitate with ethyl ether or dichloromethane to obtain the compound (0.89 g, 32% yield).); mp 240-241° C.; ¹H-NMR (300 MHz, DMSO-d₆) δ: 1.44 (3H, t, J=7.2 Hz), 3.19 (3H, s), 4.75 (1H, q, J=7.2 Hz), 7.78 (1H, t, J=8.4 Hz), 7.88 (1H, t, J=8.4 Hz), 8.32 (1H, d, J=8.4 Hz), 8.43 (1H, d, J=8.4 Hz), FAB-MS m/z (%): 178 (M⁺-Br, 19%).

Compound 2 was then used as the key starting material to react with the respective benzaldehydes to synthesize four heterocyclic resveratrol analogs 3-6 (FIG. 1).

### Example 2

### Synthesis of Compound 3

### N-Ethyl-2-[(E)-2-(2′-hydroxy-4′-methoxyphenyl)vinyl]benzothiazolium bromide

To 15 ml of n-propanol was added 258 mg (1 mmol) of 2 and 228 mg (1.5 mmol) of 2-hydroxy-4-methoxybenzaldehyde, and catalytic amount of zinc chloride. The mixture was refluxed for 3 h. At the completion of the reaction, 30 ml of ether/hexane (1/1, v/v) was added to the solution to precipitate the crude product. H₂O/MeOH/acetone (1/1/2, v/v/v) was used to recrystallize highly purified product. Bricky red powder; yield 0.36 g (92%); mp 168-170° C.; ¹H-NMR (300 MHz, DMSO-d₆) δ: 1.44 (3H, t, J=7.2 Hz), 3.81 (3H, s), 4.83 (1H, q, J=7.2 Hz), 6.52 (1H, d, J=2.1 Hz), 6.60 (1H, dd, J=8.7, 2.1 Hz), 7.23 (1H, t, J=7.2 Hz), 7.81 (1H, d, J=15.6 Hz), 7.82 (1H, t, J=7.2 Hz), 8.00 (1H, d, J=8.7 Hz), 8.23 (1H, d, J=15.6 Hz), 8.24 (1H, d, J=8.7 Hz), 8.33 (1H, d, J=8.1 Hz), 10.59 (1H, s); IR (KBr) cm⁻¹: 3150 (br, OH), 1597, 1573, 1513, 1440; FAB-MS m/z (%): 313 (M⁺-Br, 27%). Anal. (C₁₈H₁₈BrNO₂S.3.5H₂O) C, H, N, S.

### Example 3

### Synthesis of Compound 4

### N-Ethyl-2-[(E)-2-(3′,4′,5′-trimethoxyphenyl)vinyl]benzothiazolium bromide

The same method for 3 was used except 2-hydroxy-4-methoxybenzaldehyde was replaced by 247.5 mg (1.5 mmol) of 3,4,5-trimethoxybenzaldehyde. Earth yellow powder; yield 0.41 g (94%); mp 240-242° C.; ¹H-NMR (300 MHz, DMSO-d₆) δ: 1.48 (3H, t, J=7.2 Hz), 3.77 (3H, s), 3.90 (6H, s), 5.00 (1H, q, J=7.2 Hz), 7.42 (2H, s), 7.79 (1H, t, J=8.1 Hz), 7.88 (1H, t, J=8.1 Hz), 7.93 (1H, d, J=15.9 Hz), 8.19 (1H, d, J=15.9 Hz), 8.30 (1H, d, J=8.7 Hz), 8.45 (1H, d, J=8.7 Hz); IR (KBr) cm⁻¹: 3421 (br, OH), 1610, 1576, 1503, 1452. FAB-MS m/z (%):357 (M⁺-Br, 57%). Anal. (C₂₀H₂₂BrNO₃S.3H₂O) C, H, N, S.

### Example 4

### Synthesis of Compound 5

### N-Ethyl-2-[(E)-2-(3′,5′-dimethoxy-4′-hydroxyphenyl)vinyl]benzothiazolium bromide

The same method for 3 was used except 2-hydroxy-4-methoxybenzaldehyde was replaced by 273 mg (1.5 mmol) of 3,5-dimethoxy-4-hydroxybenzaldehyde (mmol). Scarlet powder; yield 0.32 g (89%); mp 238-240° C. ¹H-NMR (300 MHz, DMSO-d₆) δ: 1.46 (3H, t, J=6.9 Hz), 3.88 (6H, s), 4.95 (1H, q, J=6.9 Hz), 7.41 (2H, s), 7.75 (1H, t, J=7.2 Hz), 7.80 (1H, d, J=15.9 Hz), 7.84 (1H, t, J=7.2 Hz), 8.15 (1H, d, J=15.9 Hz), 8.24 (1H, d, J=8.1 Hz), 8.40 (1H, d, J=8.1 Hz), 9.75 (1H, s); IR (KBr) cm⁻¹: 3427 (br, OH), 1585, 1508, 1443. FAB-MS m/z (%): 343 (MtBr, 48%). Anal. (C₁₉H₂₀BrNO₃S.3H₂O) C, H, N, S.

### Example 5

### Synthesis of Compound 6

### N-Ethyl-2-[(E)-2-(4′-hydroxy-3′-methoxyphenyl)vinyl]benzothiazolium bromide

The same method for 2 was used except 2-hydroxy-4-methoxybenzaldehyde was replaced by 228 mg (1.5 mmol) of 3-methoxy-4-hydroxybenzaldehyde. Brick red powder; yield 0.35 g (89%); mp 148-150° C. ¹H-NMR (300 MHz, DMSO-d₆) δ: 1.46 (3H, t, J=7.2 Hz), 3.83 (3H, s), 4.94 (1H, q, J=7.2 Hz), 6.94 (1H, d, J=8.1 Hz), 7.57 (1H, dd, J=8.1, 2.1 Hz), 7.66 (1H, d, J=2.1 Hz), 7.75 (1H, t, J=7.5 Hz), 7.80 (1H, d, J=15.3 Hz), 7.84 (1H, t, J=7.5 Hz), 8.15 (1H, d, J=15.3 Hz), 8.24 (1H, d, J=8.4 Hz), 8.39 (1H, d, J=7.8 Hz), 10.29 (1H, s); IR (KBr) cm⁻¹: 3434 (br, OH), 1583, 1511, 1440. FAB-MS m/z (%):313 (MtBr, 43%). Anal. (C₁₈H₁₈BrNO₂S.4H₂O) C, H, N, S.

### In-vitro Biological Studies

### Example 6

### Nitric Oxide Inhibition

The mouse BALB/c macrophage cell line, RAW 264.7 BCRC No. 60001 (identical to ATCC number TIB-71), was obtained from Bioresource Collection and Research Center, Taiwan. The cells were maintained according to the protocols being cultured in 50 cm² plastic flasks (Nunc, Roskilde, Denmark) with the medium renewed every 3 days. LPS (from Escherichia coli, serotype 0127:B8), trypan blue and all the other chemicals, unless otherwise specified, were purchased from Aldrich-Sigma Chemical Company (St. Louis, Mo., USA).

### Determination of Cell Viability by an MTT Assay.

To evaluate the cell viability, a methylthiazoletetrazolium bromide (MTT) assay was conducted by the standard method in our laboratory. Incubation was performed after pretreating with a combination of the test compounds, in stock concentrations of 25, 50, 100, and up to 200 μM in dimethyl sulfoxide, and LPS (100 ng/ml) in normal saline for 24 h. Untreated cells were used as the control.

### Nitrite Quantification.

The cells were cultured with or without a pretreatment by the test compounds and resveratrol of 25, 50, 100, and up to 200 μM as already described. Each value is the mean±SEM of three determinations. *p<0.05, each value was compared with or without the LPS-stimulated group.

The production of NO was determined by measuring the accumulated level of nitrite in the culture supernatant with the Griess reagent in LPS-stimulated macrophage cells. A quantity of 100 μl of a sample aliquot were mixed with 100 μl of the Griess reagent (0.1% N-(1-naphthyl)ethylenediamine, 1% sulfanilamide, and 2.5% phosphoric acid) in a 96-well plate and then incubated at 25° C. for 10 min. The absorbance at 550 nm was measured with an ELISA reader (MR 700, Dynatech Laboratories, Alexandria, Va., USA). NaNO₂ was used as the standard to calculate the nitrite concentration.

### Results

These analogs 3-6 compared to resveratrol were evaluated for the inhibition of nitric oxide (NO) based on the accumulation of nitrite by activated macrophages in vitro. The results of NO inhibition in vitro as indicated by the reduction of nitrite are shown in Table 1. The results indicated that, under current experimental condition, all the four compounds showed better inhibition on NO production below at a cut-off concentration (25 μM) in vitro as compared to resveratrol (1) (40 μM), a natural polyphenol.

TABLE 1
IC₅₀ values of in vitro anti-angiogenesis activity and NO

| | Anti-angiogenesis, | NO inhibition, |
|---|---|---|
| Compound | IC₅₀ (μM) | IC₅₀ (μM) |
| Control (Medium only) | N/A | N/A |
| Resveratrol (1) | 47.7 ± 1.1 | 40 |
| 3 | 31.2 ± 2.0 | <25 |
| 4 | 18.8 ± 1.3 | <25 |
| 5 | 21.2 ± 1.6 | <25 |
| 6 | 19.9 ± 2.3 | <25 |

### Example 7

### Anti-Angiogenesis Study

Capillary-tube formation was assayed using the angiogenesis kits (Kurabo, Okayama, Japan) according to the manufacturer's instruction. Briefly, human umbilical vein endothelial cells (HUVECs) co-cultured with normal human dermal fibroblasts (NHDF), which a cultural kit originally developed by Kurabo Co. Ltd., Okayama, Japan, onto 24-well plate were incubated either with or without test compounds and resveratrol respectively at appropriate concentrations (1-100 μM) for 11 d (media replacement was performed at d 1, 4, 7, and 9). The cells were thereafter fixed with 70% cold ethanol, incubated with mouse antihuman CD31 antibody followed by incubation with an alkaline phosphatase conjugate goat antimouse antibody, and then stained with 5-bromo-4-chloro-3-indolyl phosphate/nitro blue tetrazolium to visualize tube network at d-11, respectively. In each assay, randomly selected five fields of view in each well were captured by digital camera under the light microscopy on ×40 magnification. The analysis of tube formation was then carried out by measuring total area and length of tubes per well using analytical software (by Kurabo).

### Results

As an initial screen for biological activity, compounds 3-6 were evaluated for anti-angiogenesis in vitro against resveratrol. The IC₅₀ values of anti-angiogenesis in vitro (by Kurabo software) were shown in Table 1. Representative images of the anti-angiogenesis were shown in FIG. 2.

The results indicate that, under current experimental condition, all four resveratrol analogs are more effective anti-angiogenic agents in vitro as compared to resveratrol (1) (IC₅₀ at 47.7±1.1 μM for HUVEC tube formation inhibition); analogs 4, 5 and 6 (comparable IC₅₀=18.8±1.3, 21.2±1.6, and 19.9±2.3 μM, respectively) appeared to be better than analog 3 (31.2±2.0 μM).

### Example 8

### In-vivo Carrageenan-Induced Paw Edema Study

In this study, X-carrageenan was used to stimulate the inflammation on the rear footpad of the rats. Male albino Wistar rats (183-218 g) 4 to 6 weeks old were housed and cared under the guidelines of the Institutional Animal Care and Use Committee at the National Defense Medical Center, Taiwan. The rats were assigned to groups. For each group, 3 rats were used and marked on the tails. one of them being the control. In order to induce inflammation, 50 μl of a 1% λ-carrageenan solution in normal saline was injected into the right hind paw subplantar tissue. The development of paw edema was measured plethysmographically (Basile 7140 plethysmometer, Ugo, Varese, Italy) and recorded prior to this administration. One hour before the λ-carrageenan challenge, a sample preparation (20 mg/kg in CMC) includes resveratrol (1) and its analogs 3, 4, 5 and 6. Before carrageenan treatment, the volume of each left footpad was measured (V₀). Each rat was injected i.p. with the followings: (1) Control Group: medium only; (2) Testing Groups (one group for each compound), compound suspension in carboxymethyl cellulose (CMC) (20 mg/kg), and (3) comparison group: resveratrol suspension in CMC (20 mg/kg) was injected i.p. into the rat in the test group. After the λ-carrageenan challenge, each paw volume (ml) was measured hourly up to 5 h. The percentage of paw edema and the inhibition of inflammation were calculated. Edema rate (E %) was calculated as follows: E%=(Vₜ-V₀)/V₀×100; V₀: volume of hind paw before 1% λ-carrageenan administration; Vₜ: volume of hind paw after 1% carrageenan administration at t h. Percentage of inhibition (1%) was determined as follows: 1%(E_{c}-Eₜ)/E_{c}×100; E_{c}: edema rate of control group; Eₜ: edema rate of the respective test compound at t h.

### Results

A state of local acute inflammation was evoked by injecting 1% (w/v) λ-carrageenan (0.1 ml/paw) s.c. into the plantar surface of the right hind paw of the rat, with the left paw (CMC treated) serving as a control. These analogs of resveratrol were evaluated for their in vivo anti-inflammatory activity at a single dose of 20 mg/kg using the carageenan-induced paw edema method in rats. In the vehicle-treated control group, the mean volume of the right hind paws increased by 0.71±0.07 ml equivalent to 82.8% edema at 5 h after a carrageenan challenge (FIG. 3). Our results obviously showed that the synthetic resveratrol analogs 3-6 exerted a striking anti-inflammatory effect with a significant reduction in swelling compared to resveratrol at rats. Among them, compound 4 manifested the greatest inhibition of 69.5% (paw edema, 0.21±0.06 ml, p<0.05) after 5 h.

Based on the above, it is pleasantly surprising that the insertion of a heterocyclic benzothiazolium moiety into the stilbene core resulted in superior biological activities. Therefore, we hereby disclose a compound having a general structure shown below

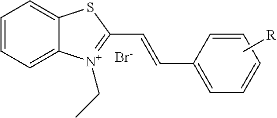

wherein -R is mono-, di- or tri-substituted —OH and/or —OMe group(s) independently.

Also disclosed in the present invention is a method of preparing a pharmaceutical composition which comprises the compound with the general structure shown and suitable pharmaceutical excipients wherein the composition is made by a standard pharmaceutical preparation method. We also disclose a method to treat angiogenesis- or inflammation-related disorder which comprises administering a therapeutically effective amount of the above mentioned pharmaceutical composition to a patient; the angiogenesis-related disorder includes cancer and inflammation-related disorder includes atherosclerosis, arthritis, type 1 diabetes, Crohn's disease, and allergies.

### SUMMARY, RAMIFICATION, AND SCOPE

In conclusion, the present invention discloses a series of novel analogs of resveratrol. The results of all studies to date indicate these analogs are surprisingly better than resveratrol pharmacologically. Also disclosed in the present invention is a method of treating for treating disorders related to angiogenesis and inflammation.

Although the description above contains many specificities, these should not be construed as limiting the scope of the invention but as merely providing the illustrations of some of the presently preferred embodiments of this invention. Thus the scope of this invention should be determined by the appended claims and their legal equivalents, rather than by the examples given.

### DRAWINGS

FIG. 1 shows the structures of resveratrol, (E)-stilbene, and the synthetic scheme of compounds 3-6. The reaction conditions are also described in the synthetic scheme.

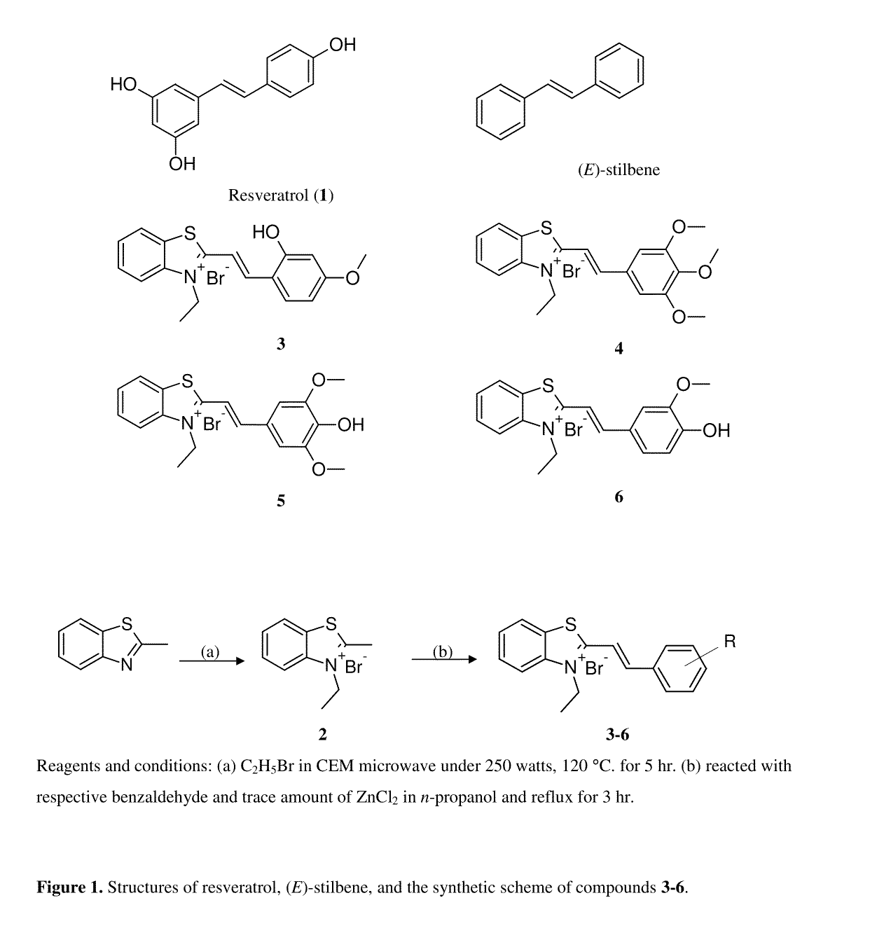

FIG. 2 shows the representative images of the anti-angiogenesis (40×) by the test compounds.

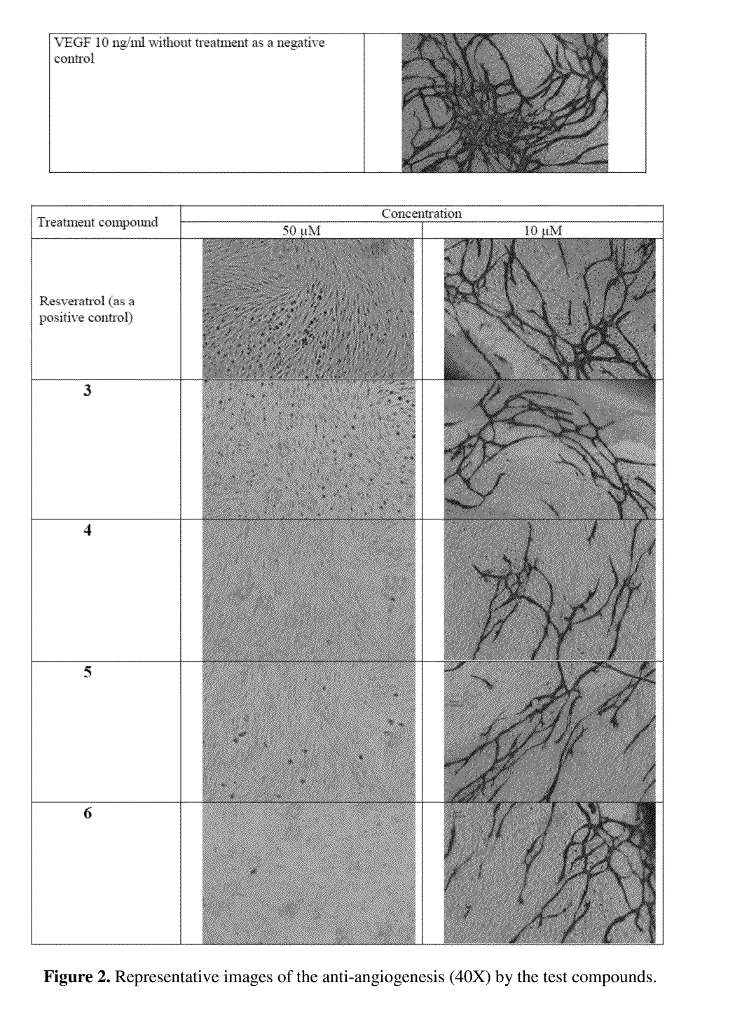

FIG. 3 shows the anti-inflammatory activities of compounds 3-6 at 20 mg/kg i.p. single dose in carrageenan-induced paw edema model in rats (n=3 animals/group). Each value represents the average±SEM of 3 animals.

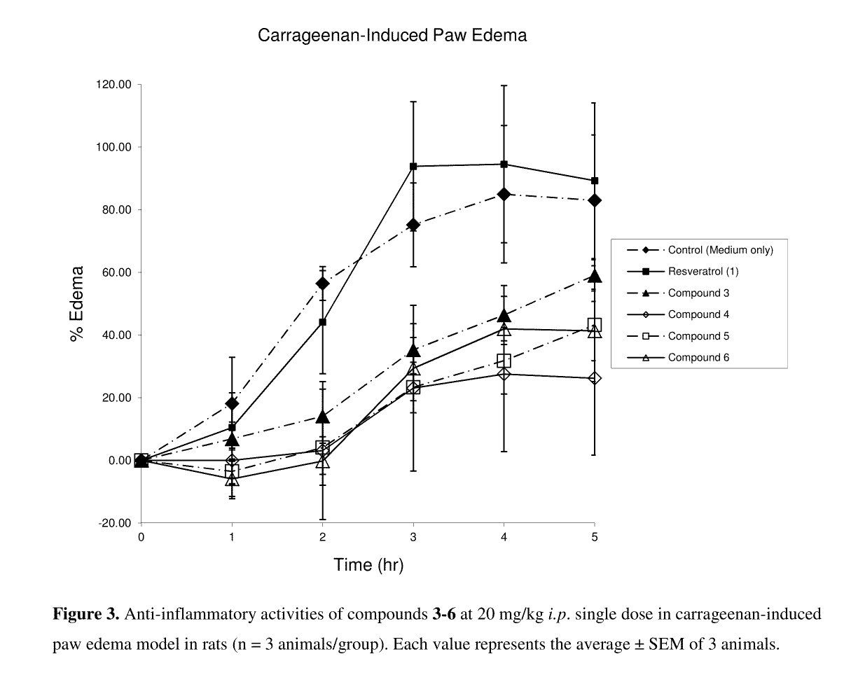

1. A compound having a general structure shown below 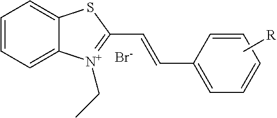
wherein -R is mono-, di- or tri-substituted —OH and/or —OMe group(s) independently.

2. A method of preparing a pharmaceutical composition which comprises the compound according to claim 1 and suitable pharmaceutical excipients wherein the composition is made by a standard pharmaceutical preparation method.

3. A method to treat angiogenesis- or inflammation-related disorder which comprises administering a therapeutically effective amount of the pharmaceutical composition according to claim 2 to a patient.

4. The method according to claim 3 wherein the angiogenesis-related disorder includes cancer.

5. The method according to claim 3, wherein inflammation-related disorder includes atherosclerosis, arthritis, type 1 diabetes, Crohn's disease, and allergies.
